(19) United States
(12) Patent Application Publication
(10) Pub. No.: US 2021/0068723 A1
(43) Pub. Date: Mar. 11, 2021

(54) INTELLIGENT PREDICTION-BASED GLUCOSE ALARM DEVICES, SYSTEMS, AND METHODS

(71) Applicant: MEDTRONIC MINIMED, INC., Northridge, CA (US)

(72) Inventors: Ayca Balkan, Los Angeles, CA (US); Benyamin Grosman, Valley Village, CA (US); Maria Diana Miller, Santa Rosa Valley, CA (US); Dmytro Y. Sokolovskyy, Moorpark, CA (US)

(21) Appl. No.: 16/565,997

(22) Filed: Sep. 10, 2019

(51) Int. Cl. A61B 5/145; G16H 20/17; G16H 50/30; G16H 50/20; G16H 80/00; A61B 5/00

(52) U.S. Cl. CPC A61B 5/14532; G16H 20/17; A61B 5/746; G16H 50/20; G16H 80/00; G16H 50/30

(57) ABSTRACT

Glucose-related alarms may be generated based on glucose predictions, which may be short-to-medium term. The glucose predictions, in turn, may be computed based on a multiplicity of input data sources which may be indicative of the current and future states of the user, as well as the user's infusion pump and overall glucose management system. The input data may include, e.g., meal information, motion/exercise information, insulin information, glucose, and pump- and sensor-related metrics. In addition, the computed glucose predictions may be based on the system's expected insulin-delivery response. The computed glucose predictions inform the determination as to whether, when, and how, to sound a certain glucose alarm, or display a certain glucose alert to the user.

### FIELD

The present technology is generally related to sensor technology, including sensors used for sensing glucose concentration, and to enabling generation of glucose-related alarms based on glucose predictions.

### BACKGROUND

Over the years, a variety of sensors have been developed for detecting and/or quantifying specific agents or compositions in a patient's blood, which enable patients and medical personnel to monitor physiological conditions within the patient's body. Illustratively, subjects may wish to monitor blood glucose levels in a subject's body on a continuing basis. Thus, glucose sensors have been developed for use in obtaining an indication of blood glucose levels in a diabetic patient. Such readings are useful in monitoring and/or adjusting a treatment regimen which typically includes the regular administration of insulin to a patient.

Traditionally, patients have measured their blood glucose (BG) using a BG measurement device (i.e., glucose meter), such as a test strip meter, a continuous glucose measurement system (or a continuous glucose monitor), or a hospital hemacue. BG measurement devices use various methods to measure the BG level of a patient, such as a sample of the patient's blood, a sensor in contact with a bodily fluid, an optical sensor, an enzymatic sensor, or a fluorescent sensor. When the BG measurement device has generated a BG measurement, the measurement may be displayed on the BG measurement device.

Current continuous glucose measurement systems include subcutaneous (or short-term) sensors and implantable (or long-term) sensors. Sensors have been applied in a telemetered characteristic monitor system. A telemetered system using an electrochemical sensor may include a remotely located data receiving device, a sensor for producing signals indicative of a characteristic of a user, and a transmitter device for processing signals received from the sensor and for wirelessly transmitting the processed signals to the remotely located data receiving device. The data receiving device may be a characteristic monitor, a data receiver that provides data to another device, an RF programmer, a medication delivery device (such as an infusion pump), or the like.

Regardless of whether the data receiving device (e.g., a glucose monitor), the transmitter device, and the sensor (e.g., a glucose sensor) communicate wirelessly or via an electrical wire connection, a characteristic monitoring system of the type described above is of practical use only after it has been calibrated based on the unique characteristics of the individual user. In some current devices, the patient/user may be required to externally calibrate the sensor. More specifically, and in connection with the illustrative example of a diabetic patient, the latter may be required to utilize a finger-stick blood glucose meter reading an average of two-four times per day for the duration that the characteristic monitor system is used. Each time, blood is drawn from the user's finger and analyzed by the blood glucose meter to provide a real-time blood sugar level for the user. The user then inputs this data into the glucose monitor as the user's current blood sugar level which is used to calibrate the glucose monitoring system.

Such external calibrations, however, are disadvantageous for various reasons. For example, blood glucose meters are not perfectly accurate and include inherent margins of error. Moreover, even if completely accurate, blood glucose meters are susceptible to improper use; for example, if the user has handled candy or other sugar-containing substance immediately prior to performing the finger stick, with some of the sugar sticking to the user's fingers, the blood sugar analysis will result in an inaccurate blood sugar level indication. Furthermore, there is a cost, not to mention pain and discomfort, associated with each application of the finger stick.

The current state of the art in continuous glucose monitoring (CGM) is largely adjunctive, meaning that the readings provided by a CGM device (including, e.g., an implantable or subcutaneous sensor) cannot be used without a reference value in order to make a clinical decision. The reference value, in turn, must be obtained from a finger stick using, e.g., a BG meter. The reference value is needed because there is a limited amount of information that is available from the sensor/sensing component. Specifically, the only pieces of information that are currently provided by the sensing component for processing are the raw sensor value (i.e., the sensor current or Isig) and the counter voltage. Therefore, during analysis, if it appears that the raw sensor signal is abnormal (e.g., if the signal is decreasing), the only way one can distinguish between a sensor failure and a physiological change within the user/patient (i.e., glucose level changing in the body) is by acquiring a reference glucose value via a finger stick. As is known, the reference finger stick is also used for calibrating the sensor.

The art has searched for ways to eliminate or, at the very least, minimize, the number of finger sticks that are necessary for calibration and for assessing sensor health. Here, diagnostics have been developed that are based on either direct assessment of the Isig, or on comparison of multiple Isigs, e.g., from redundant and/or orthogonally redundant, sensors and/or electrodes. In either case, because the Isig tracks the level of glucose in the body, by definition, it is not analyte independent. As such, by itself, the Isig is not a reliable source of information for sensor diagnostics, nor is it a reliable predictor for continued sensor performance.

The art has also searched for more accurate and reliable means for providing self-calibrating sensors, and for performing sensor diagnostics by developing a variety of circuit models. In such models, an attempt is generally made to correlate circuit elements to parameters that may be used in intelligent diagnostics, gross failure analysis, and real-time self-calibrations using, e.g., electrochemical impedance spectroscopy (EIS).

### SUMMARY

In one aspect, the present disclosure provides a method of providing a real-time alert to a user of a glucose management system, the system including an infusion device, a glucose sensor used for measuring the level of glucose in the user's body, physical sensor electronics, and a microcontroller, the method comprising: (a) periodically measuring, by the physical sensor electronics, electrode current (Isig) signals indicative of the level of glucose in the user's body; (b) generating, by the microcontroller, a predicted glucose profile for the user based on the level of glucose in the user's body and based on meal information, activity information, and insulin information; (c) determining in real time, by the microcontroller, whether the predicted glucose profile is expected to reach an acceptable level within an expected time window; (d) determining in real time, by the microcontroller, whether an alarm should be generated to alert the user if the microcontroller determines that the predicted glucose profile is not expected to reach the acceptable level within the expected time window; and (e) providing the alert to the user as a real-time alarm if the predicted glucose profile is not expected to reach the acceptable level within said expected time window.

In another aspect, the disclosure provides an insulin infusion pump comprising a housing; an alarm module within the housing; a display on the housing; and a controller that is configured to provide a real-time alert to a user of the insulin infusion pump, the pump being in operative communication with a glucose sensor used for measuring the level of glucose in the user's body, wherein the controller periodically receives electrode current (Isig) signals indicative of the level of glucose in the user's body; generates a predicted glucose profile for the user based on the level of glucose in the user's body and based on meal information, activity information, and insulin information; determines, in real time, whether the predicted glucose profile is expected to reach an acceptable level within an expected time window; determines, in real time, whether an alarm should be generated to alert the user if the controller determines that the predicted glucose profile is not expected to reach the acceptable level within the expected time window; and provides the alert to the user as a real-time alarm if the predicted glucose profile is not expected to reach the acceptable level within the expected time window.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of embodiments of the invention will be made with reference to the accompanying drawings, wherein like numerals designate corresponding parts in the figures.

FIG. 1 is a perspective view of a subcutaneous sensor insertion set and block diagram of a sensor electronics device.

FIG. 2A illustrates a substrate having two sides, a first side which contains an electrode configuration and a second side which contains electronic circuitry.

FIG. 2B illustrates a general block diagram of an electronic circuit for sensing an output of a sensor.

FIG. 3 illustrates a block diagram of a sensor electronics device and a sensor including a plurality of electrodes according.

FIG. 4 illustrates an alternative diagram including a sensor and a sensor electronics device.

FIG. 5 illustrates an electronic block diagram of the sensor electrodes and a voltage being applied to the sensor electrodes.

FIG. 6 shows a flow diagram and system in accordance with embodiments of the invention.

FIG. 7 shows a matrix of level of severity of user state vs. the amount of time spent by the user in a given state, in accordance with embodiments of the invention.

### DETAILED DESCRIPTION

In the following description, reference is made to the accompanying drawings which form a part hereof and which illustrate embodiments of the present inventions. It is understood that other embodiments may be utilized, and structural and operational changes may be made without departing from the scope of the present inventions.

The inventions herein may be described below with reference to flowchart illustrations of methods, systems, devices, apparatus, and programming and computer program products. It will be understood that each block of the flowchart illustrations, and combinations of blocks in the flowchart illustrations, can be implemented by programing instructions, including computer program instructions (as can any menu screens described in the figures). These computer program instructions may be loaded onto a computer or other programmable data processing apparatus (such as a controller, microcontroller, or processor in a sensor electronics device) to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks, and/or menus presented herein. Programming instructions may also be stored in and/or implemented via electronic circuitry, including integrated circuits (ICs) and Application Specific Integrated Circuits (ASICs) used in conjunction with sensor devices, apparatuses, and systems.

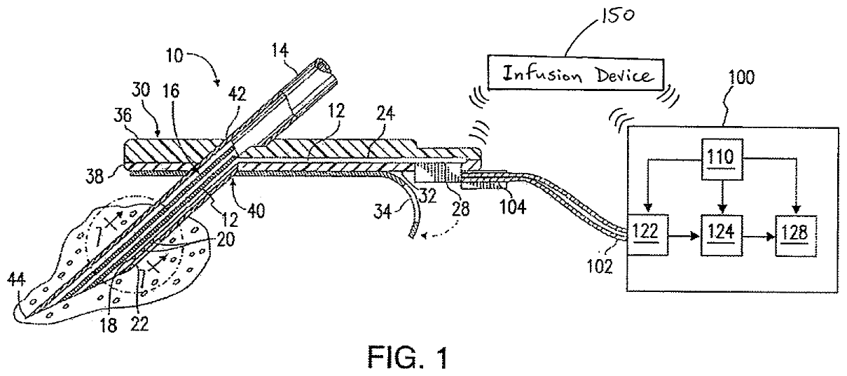

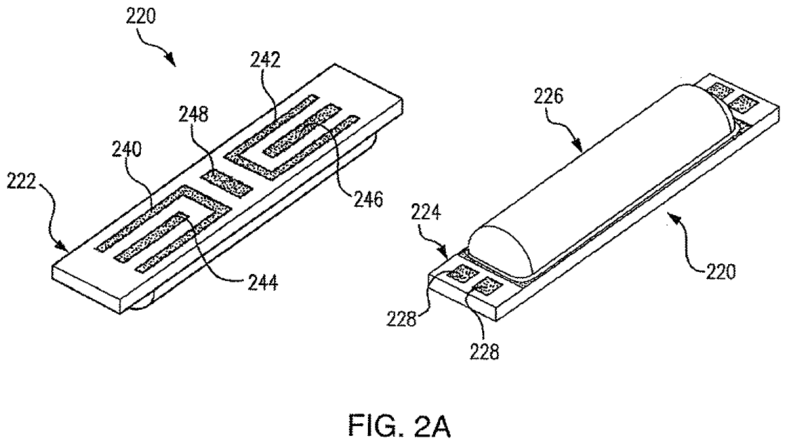

FIG. 1 is a perspective view of a subcutaneous sensor insertion set and a block diagram of a sensor electronics device. As illustrated in FIG. 1, a subcutaneous sensor set 10 is provided for subcutaneous placement of an active portion of a flexible sensor 12 (see, e.g., FIG. 2), or the like, at a selected site in the body of a user. The subcutaneous or percutaneous portion of the sensor set 10 may include a hollow, slotted insertion needle 14, and a cannula 16. The needle 14 is used to facilitate quick and easy subcutaneous placement of the cannula 16 at the subcutaneous insertion site. Inside the cannula 16 is a sensing portion 18 of the sensor 12 to expose one or more sensor electrodes 20 to the user's bodily fluids through a window 22 formed in the cannula 16. In an embodiment of the invention, the one or more sensor electrodes 20 may include a counter electrode, a reference electrode, and one or more working electrodes. After insertion, the insertion needle 14 is withdrawn to leave the cannula 16 with the sensing portion 18 and the sensor electrodes 20 in place at the selected insertion site.

In particular embodiments, the subcutaneous sensor set 10 facilitates accurate placement of a flexible thin film electrochemical sensor 12 of the type used for monitoring specific blood parameters representative of a user's condition. The sensor 12 monitors glucose levels in the body and may be used in conjunction with automated or semi-automated medication infusion pumps of the external or implantable type as described, e.g., in U.S. Pat. Nos. 4,562,751, 4,678,408, 4,685,903, or 4,573,994, and in particular embodiments, in a closed-loop system, to control delivery of insulin to a diabetic patient.

Particular embodiments of the flexible electrochemical sensor 12 may be constructed in accordance with thin film mask techniques to include elongated thin film conductors embedded or encased between layers of a selected insulative material such as polyimide film or sheet, and membranes. The sensor electrodes 20 at a tip end of the sensing portion 18 are exposed through one of the insulative layers for direct contact with patient blood or other body fluids, when the sensing portion 18 (or active portion) of the sensor 12 is subcutaneously placed at an insertion site. The sensing portion 18 is joined to a connection portion 24 that terminates in conductive contact pads, or the like, which are also exposed through one of the insulative layers. In alternative embodiments, other types of implantable sensors, such as chemical based, optical based, or the like, may be used.

As is known in the art, the connection portion 24 and the contact pads are generally adapted for a direct wired electrical connection to a suitable monitor or sensor electronics device 100 for monitoring a user's condition in response to signals derived from the sensor electrodes 20. Further description of flexible thin film sensors of this general type may be found, e.g., in U.S. Pat. No. 5,391,250, which is incorporated herein by reference. The connection portion 24 may be conveniently connected electrically to the monitor or sensor electronics device 100 or by a connector block 28 (or the like) as shown and described, e.g., in U.S. Pat. No. 5,482,473, which is also incorporated herein by reference. Thus, in accordance with embodiments, subcutaneous sensor sets 10 may be configured or formed to work with either a wired or a wireless characteristic monitor system.

The sensor electrodes 20 may be used in a variety of sensing applications and may be configured in a variety of ways. For example, the sensor electrodes 20 may be used in physiological parameter sensing applications in which some type of biomolecule is used as a catalytic agent. For example, the sensor electrodes 20 may be used in a glucose and oxygen sensor having a glucose oxidase (GOx) enzyme catalyzing a reaction with the sensor electrodes 20. The sensor electrodes 20, along with a biomolecule or some other catalytic agent, may be placed in a human body in a vascular or non-vascular environment. For example, the sensor electrodes 20 and biomolecule may be placed in a vein and be subjected to a blood stream or may be placed in a subcutaneous or peritoneal region of the human body.

The monitor 100 may also be referred to as a sensor electronics device 100. The monitor 100 may include a power source 110, a sensor interface 122, processing electronics 124, and data formatting electronics 128. The monitor 100 may be coupled to the sensor set 10 by a cable 102 through a connector that is electrically coupled to the connector block 28 of the connection portion 24. In an alternative embodiment, the cable may be omitted. In this embodiment, the monitor 100 may include an appropriate connector for direct connection to the connection portion 104 of the sensor set 10. The sensor set 10 may be modified to have the connector portion 104 positioned at a different location, e.g., on top of the sensor set to facilitate placement of the monitor 100 over the sensor set.

In embodiments, the sensor interface 122, the processing electronics 124, and the data formatting electronics 128 may be formed as separate semiconductor chips; however, alternative embodiments may combine the various semiconductor chips into a single, or multiple customized semiconductor chips. The sensor interface 122 may connect with the cable 102 that is connected with the sensor set 10.

The power source 110 may be a battery. The battery can include, e.g., a multiplicity of silver oxide 357 battery cells. In alternative embodiments, different battery chemistries may be utilized, such as lithium-based chemistries, alkaline batteries, nickel metalhydride, or the like, and a different number of batteries may be used. In one embodiment, the monitor 100 provides power to the sensor set via the power source 110, through the cable 102 and cable connector 104. The power may be, e.g., a voltage or a current that is provided to the sensor set 10. In other embodiments, the sensor set 10 may have its own power source and/or electronics, and may communicate wirelessly with the monitor 100 and/or an infusion device, such as, e.g., an insulin pump 150, having its own controller(s), software, and/or electronics. Similarly, the monitor 100 may communicate wirelessly with the infusion device. Together, the foregoing enable various configurations of a closed-loop, or semi-closed-loop, continuous glucose monitoring system.

FIGS. 2A and 2B illustrate an implantable sensor, and electronics for driving the implantable sensor in accordance with one embodiment. FIG. 2A shows a substrate 220 having two sides, a first side 222 of which contains an electrode configuration and a second side 224 of which contains electronic circuitry. As may be seen in FIG. 2A, a first side 222 of the substrate comprises two counter electrode-working electrode pairs 240, 242, 244, 246 on opposite sides of a reference electrode 248. A second side 224 of the substrate comprises electronic circuitry. As shown, the electronic circuitry may be enclosed in a hermetically sealed casing 226, providing a protective housing for the electronic circuitry. This allows the sensor substrate 220 to be inserted into a vascular environment or other environment which may subject the electronic circuitry to fluids. By sealing the electronic circuitry in a hermetically sealed casing 226, the electronic circuitry may operate without risk of short circuiting by the surrounding fluids. Also shown in FIG. 2A are pads 228 to which the input and output lines of the electronic circuitry may be connected. The electronic circuitry itself may be fabricated in a variety of ways. According to an embodiment, the electronic circuitry may be fabricated as an integrated circuit using techniques common in the industry.

FIG. 2B illustrates a general block diagram of an electronic circuit for sensing an output of a sensor. In this configuration, at least one pair of sensor electrodes 310 may interface to a data converter 312, the output of which may interface to a counter 314. The counter 314 may be controlled by control logic 316. The output of the counter 314 may connect to a line interface 318. The line interface 318 may be connected to input and output lines 320 and may also connect to the control logic 316. The input and output lines 320 may also be connected to a power rectifier 322.

The sensor electrodes 310 may be used in a variety of sensing applications and may be configured in a variety of ways. For example, the sensor electrodes 310 may be used in physiological parameter sensing applications in which some type of biomolecule is used as a catalytic agent. For example, the sensor electrodes 310 may be used in a glucose and oxygen sensor having a glucose oxidase (GOx) enzyme catalyzing a reaction with the sensor electrodes 310. The sensor electrodes 310, along with a biomolecule or some other catalytic agent, may be placed in a human body in a vascular or non-vascular environment. For example, the sensor electrodes 310 and biomolecule may be placed in a vein and be subjected to a blood stream.

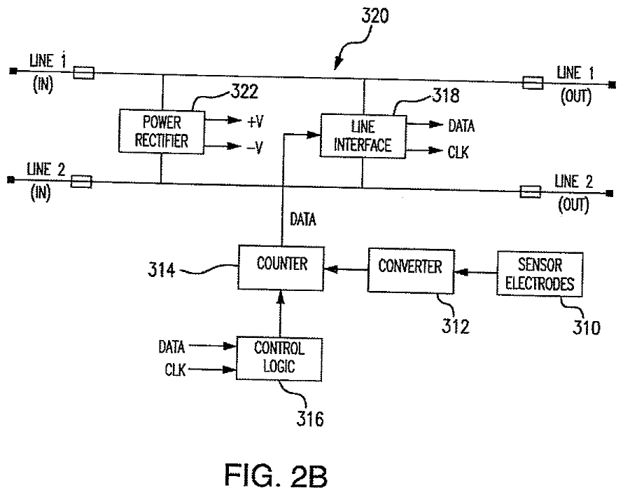

FIG. 3 illustrates a block diagram of a sensor electronics device and a sensor including a plurality of electrodes according to one embodiment. The sensor set or system 350 includes a sensor 355 and a sensor electronics device 360. The sensor 355 may include at least one of each of a counter electrode 365, a reference electrode 370, and a working electrode 375. The sensor electronics device 360 includes a power supply 380, a regulator 385, a signal processor 390, a measurement processor 395, and a display/transmission module 397. The power supply 380 provides power (e.g., in the form of either a voltage, a current, or a voltage including a current) to the regulator 385. The regulator 385 transmits a regulated voltage to the sensor 355. In an embodiment of the invention, the regulator 385 transmits a voltage to the counter electrode 365 of the sensor 355.

The sensor 355 creates a sensor signal indicative of a concentration of a physiological characteristic being measured. For example, the sensor signal may be indicative of a blood glucose reading. In an embodiment utilizing subcutaneous sensors, the sensor signal may represent a level of hydrogen peroxide in a subject. In an embodiment where blood or cranial sensors are utilized, the amount of oxygen is being measured by the sensor and is represented by the sensor signal. In an embodiment utilizing implantable or long-term sensors, the sensor signal may represent a level of oxygen in the subject. The sensor signal may be measured at the working electrode 375. In an embodiment, the sensor signal may be a current measured at the working electrode. In another embodiment, the sensor signal may be a voltage measured at the working electrode.

The signal processor 390 receives the sensor signal (e.g., a measured current or voltage) after the sensor signal is measured at the sensor 355 (e.g., the working electrode). The signal processor 390 processes the sensor signal and generates a processed sensor signal. The measurement processor 395 receives the processed sensor signal and calibrates the processed sensor signal, e.g., by utilizing reference values. In one embodiment, the reference values may be stored in a reference memory and provided to the measurement processor 395, which may generate sensor measurements. The sensor measurements may be stored in a measurement memory (not shown). The sensor measurements may be sent to a display/transmission device to be either displayed on a display in a housing with the sensor electronics, or transmitted to an external device, such as, e.g., a monitor and/or an infusion device, which may also have a display.

The sensor electronics device 360 may be a monitor which includes a display to display physiological characteristics readings. The sensor electronics device 360 may also be installed in a desktop, laptop, or network computer, a personal digital assistant (PDA), a portable/cellular telephone, a smartphone/smart device, an infusion pump including a display, a glucose sensor including a display, and/or a combination infusion pump/glucose sensor. The sensor electronics device 360 may be housed in a hand-held communications device, a network device, a home network device, or an appliance connected to a home network.

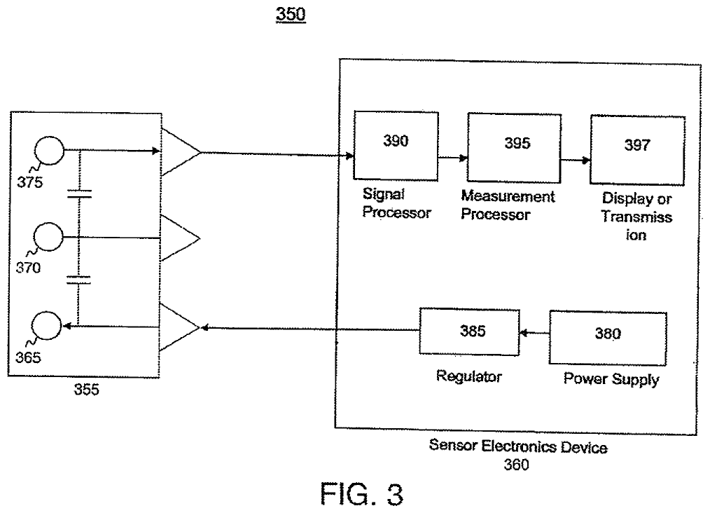

FIG. 4 illustrates an alternative embodiment including a sensor and a sensor electronics device. The sensor set or sensor system 400 may include a sensor electronics device 360 and a sensor 355. The sensor 355 may include at least one of each of a counter electrode 365, a reference electrode 370, and a working electrode 375. In this embodiment, the sensor electronics device 360 includes a microcontroller 410 and a digital-to-analog converter (DAC) 420. The sensor electronics device 360 may also include a current-to-frequency converter (I/F converter) 430.

The microcontroller 410 includes software program code, which when executed, or programmable logic which, causes the microcontroller 410 to transmit a signal to the DAC 420, where the signal is representative of a voltage level or value that is to be applied to the sensor 355. The DAC 420 receives the signal and generates the voltage value at the level instructed by the microcontroller 410. In some embodiments, the microcontroller 410 may change the representation of the voltage level in the signal frequently or infrequently. Illustratively, the signal from the microcontroller 410 may instruct the DAC 420 to apply a first voltage value for one second and a second voltage value for two seconds.

The sensor 355 may receive the voltage level or value. Thus, e.g., the counter electrode 365 may receive the output of an operational amplifier which has as inputs the reference voltage and the voltage value from the DAC 420. The application of the voltage level causes the sensor 355 to create a sensor signal indicative of a concentration of a physiological characteristic being measured. In an embodiment, the microcontroller 410 may measure the sensor signal (e.g., a current value) from the working electrode. Illustratively, a sensor signal measurement circuit 431 may measure the sensor signal. In an embodiment, the sensor signal measurement circuit 431 may include a resistor and the current may be passed through the resistor to measure the value of the sensor signal. In another embodiment, the sensor signal may be a current level signal and the sensor signal measurement circuit 431 may be a current-to-frequency (I/F) converter 430. The current-to-frequency converter 430 may measure the sensor signal in terms of a current reading, convert it to a frequency-based sensor signal, and transmit the frequency-based sensor signal to the microcontroller 410.

After the microcontroller 410 receives the sensor signal, whether frequency-based or non-frequency-based, it determines a value for the physiological characteristic of a subject, such as a blood glucose level. The microcontroller 410 may include program code, which when executed or run, is able to receive the sensor signal and convert the sensor signal to a physiological characteristic value. In an embodiment, the microcontroller 410 may convert the sensor signal to a blood glucose level. In an embodiment, the microcontroller 410 may utilize measurements stored within an internal memory in order to determine the blood glucose level of the subject. In an embodiment, the microcontroller 410 may utilize measurements stored within a memory external to the microcontroller 410 to assist in determining the blood glucose level of the subject.

After the physiological characteristic value is determined by the microcontroller 410, the microcontroller 410 may store measurements of the physiological characteristic values for a number of time periods. For example, a blood glucose (BG) value may be sent to the microcontroller 410 from the sensor every second or every five seconds, and the microcontroller may save sensor measurements for, e.g., five minutes or ten minutes of BG readings. The microcontroller 410 may transfer the measurements of the physiological characteristic values to a display on the sensor electronics device 360. For example, the sensor electronics device 360 may be a monitor which includes a display that provides a blood glucose reading for a subject. In an embodiment, the microcontroller 410 may transfer the measurements of the physiological characteristic values to an output interface of the microcontroller 410. The output interface of the microcontroller 410 may transfer the measurements of the physiological characteristic values, e.g., blood glucose values, to an external device, e.g., an infusion pump, a combined infusion pump/glucose meter, a computer, a personal digital assistant, a pager, a network appliance, a server, a cellular phone, or any computing device, one or more of which may have a display for displaying blood glucose values, as well as glucose-related data, glucose concentration profile(s), real-time, expected, and/or predicted glucose data and/or trending, insulin-delivery data, insulin-delivery pattern(s), alarm data, (visual) alarm and/or alert indication(s) or notification(s), etc.

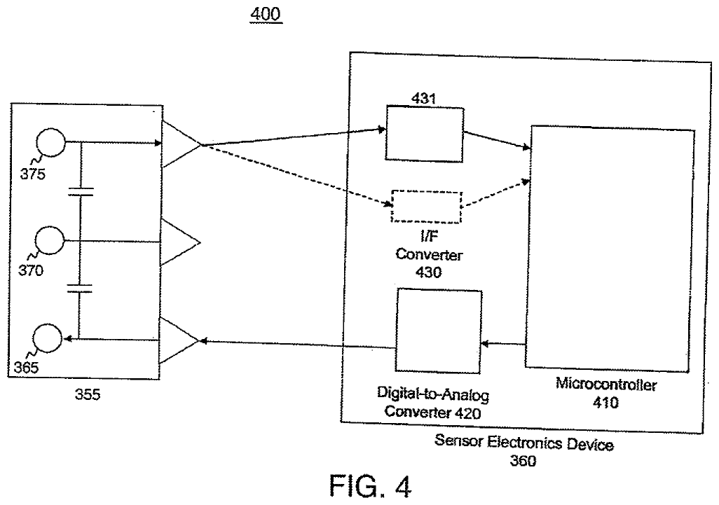

FIG. 5 illustrates an electronic block diagram of the sensor electrodes and a voltage being applied to the sensor electrodes in accordance with an embodiment. In this configuration, an op amp 530 or other servo-controlled device may connect to sensor electrodes 510 through a circuit/electrode interface 538. The op amp 530, utilizing feedback through the sensor electrodes, attempts to maintain a prescribed voltage (what the DAC may desire the applied voltage to be) between a reference electrode 532 and a working electrode 534 by adjusting the voltage at a counter electrode 536. Current may then flow from a counter electrode 536 to a working electrode 534. Such current may be measured to ascertain the electrochemical reaction between the sensor electrodes 510 and the biomolecule of a sensor that has been placed in the vicinity of the sensor electrodes 510 and used as a catalyzing agent. The circuitry disclosed in FIG. 5 may be utilized in a long-term or implantable sensor or may be utilized in a short-term or subcutaneous sensor.

Embodiments of the inventions described herein are directed to advancements and improvements in continuous glucose monitoring (CGM). In this regard, in general, a controller (e.g., of an infusion device, such as an insulin pump) is designed to model a pancreatic beta cell (β-cell). In other words, the controller commands the infusion device to release insulin into the body of a user/patient at a rate that causes the insulin concentration in the blood to follow a similar concentration profile as would be caused by fully functioning human β-cells responding to blood glucose concentrations in the body.

A controller that simulates the body's natural insulin response to blood glucose levels not only makes efficient use of insulin, but also accounts for other bodily functions as well since insulin has both metabolic and mitogenic effects. Controller algorithms that are designed to minimize glucose excursions in the body without regard for how much insulin is delivered may cause excessive weight gain, hypertension, and atherosclerosis. Similarly, algorithms that are designed to minimize glucose excursions in the body without regard for the time period over which the glucose excursion may occur, or the time period over which the insulin is delivered and/or the insulin-delivery pattern, may lead to hypoglycemia, thereby causing dizziness, or, in severe cases, death.

Insulin pumps of the type discussed herein and/or described in connection with embodiments of the inventions herein, may include an alarm system that notifies users of negative situations with their devices or their physiological states. However, in current insulin pumps, the component of this system that monitors the user's glucose is rather simple, as it only considers the user's glucose data when generating such notifications. This can often result in non-actionable alerts—where a user has already addressed the issue, or no immediate action is required—or lack of alerts when an early warning of a likely issue would have been helpful (e.g., a false negative). Thus, in one embodiment, the inventions herein include an alarm system that addresses the foregoing issues by generating short and medium-range predictions using multiple sources of data (such as, e.g., glucose, insulin, meal information, motion/exercise, etc.). The system then determines whether a prediction indicates a possible adverse condition that the system cannot address on its own, and places it in one of a plurality of categories (e.g., three) based on its severity and onset time, wherein the category determines how the user is alerted. This system can also change the category of an alert dynamically by reevaluating the predictions, allowing it to remove obsolete and irrelevant alerts and escalate the annunciation of existing alerts. This new alarm logic results in a system that can limit user alerts to only actionable ones, and can provide early warnings and longer lead times to help a user prevent adverse conditions from developing or worsening.

As noted above, in some existing systems, the approach to generating insulin pump alarms has been limited to evaluating each condition in isolation, and generating an alarm when the condition reached an unacceptable level. For glucose, for example, this approach resulted in threshold-based alarms that are generated when the user's glucose values are outside a defined range. As is known, there are valid, and generally low-risk, conditions when glucose values can be outside of a range. Nevertheless, the existing approach can result in generation of many non-actionable alarms, i.e., alarms that occur when a user is not in danger, or when a user cannot do anything to mitigate or improve the condition. For example, if the user ate a big meal and took sufficient insulin to cover it, the user's glucose values can still exceed his/her threshold for a short amount of time. This, however, is expected, and does not cause harm to the user. However, in the existing system described hereinabove, the user will still receive a high-glucose notification/alarm when the user passes his/her high glucose threshold.

In addition, any underestimation of meal carbohydrates translates into an underestimation of meal boluses. This might cause the user's glucose to exceed the high threshold even in a closed-loop delivery setting. Importantly, however, in most of such situations, there is still no required action to be taken by the user since the closed-loop algorithm will automatically correct the glucose levels by adjusting the insulin delivery. Yet, with existing alarm systems, the user will still receive high-glucose alerts (such as an alarm or other notification).

To reduce the number of non-actionable alarms, embodiments of the inventions herein predict future glucose state(s) to determine whether the user is likely to end up in an undesirable situation soon. This is accomplished by computing predictions of future glucose based on various inputs, including current and past glucose values, meal information (such as carbohydrate amounts, meal timing, and predicted meal information inferred from past meal events or from external meal event predictions (e.g., Watson/SugarIQ)), activity and exercise information (such as current activity and predicted activity inferred from past trends or from external activity predictors), insulin delivery, and current hardware state (including device battery state, sensor connection and calibration state, sensor reliability metrics, and insulin amount remaining in reservoir).

Since insulin delivery is extremely important in producing accurate glucose predictions, embodiments of the inventions herein may also consider future delivery by effectively simulating the insulin delivery behavior over the entire time horizon. In other words, the alarm/notification system can also use the expected future insulin delivery of the system based on the predicted glucose values.

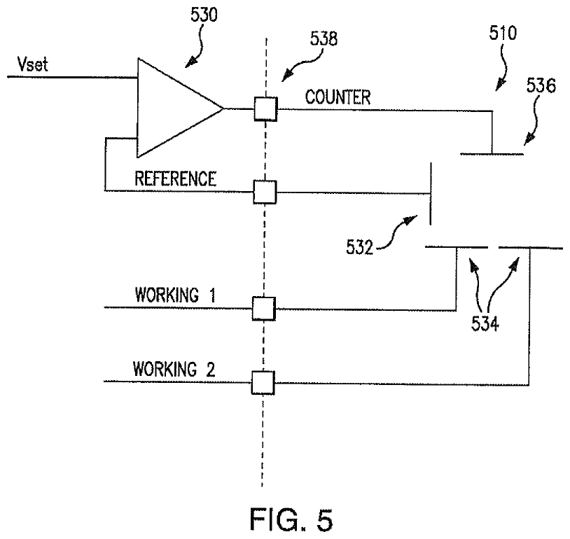

More specifically, FIG. 6 shows a flow diagram and system in which the above-described data, including glucose values, meal information, activity information, insulin delivery, and current hardware state may be used as inputs to generate predicted glucose values and/or concentration profiles via a glucose prediction module 610. The glucose prediction module 610 may use any known glucose prediction algorithm to generate the predicted glucose values/profiles. It is also understood that, in FIG. 6, each of the modules, as well as communication and transmission of data amongst the modules, may be embodied in: (1) software, such as, e.g., software in a controller or processor to operationalize the glucose prediction module (610), to carry out the insulin delivery algorithm (620), and/or to assess whether conditions for alarm generation via the alarm module (630) are met, and to generate such alarm; and/or (2) hardware, such as, e.g., an infusion pump for insulin delivery.

As shown in FIG. 6, once generated, the predicted glucose values/profiles (615) may be used as input to the insulin delivery algorithm of the insulin delivery module (620), and the alarm module (630). With regard to the former, the system can use the pre-set basal rates while computing the effect of the future insulin delivery on glucose predictions. The insulin delivery algorithm may utilize, e.g., Predictive Low Glucose Management (PLGM), Hybrid Closed Loop (HCL), and Advanced Hybrid Closed Loop (AHCL) algorithms.

In embodiments of the invention, the algorithm may be implemented on existing (continuous glucose management) CGM platforms. Thus, an embodiment may allow, e.g., for use with hybrid closed-loop (HCL) systems, while another embodiment may allow for use in stand-alone CGM systems. In the former embodiment, the system may have transmitters that communicate with an insulin pump supporting a HCL algorithm via a proprietary radio-frequency protocol. In the latter embodiment, the communication protocol may be, e.g., Bluetooth Low Energy Technology that is supported through a mobile device display application. When supporting an HCL system, the transmitter may include logic to ensure sensor values reliably support insulin dosing.

Predictive Low Glucose Management (PLGM) utilizes a next-generation integrated insulin pump and CGM system, which automatically stops insulin delivery when the sensor measures sensor glucose level predicted to approach the low limit, and then resumes insulin delivery once sensor glucose levels recover. If the system is integrated into a pump with the PLGM feature, it can take into account when, and if, the insulin delivery will be suspended by the PLGM based on the generated glucose prediction and adjust the future glucose predictions accordingly. For example, if the insulin delivery suspension will keep the user in the therapy target range, then the system does not need to alarm the user and, therefore, will not generate an alarm/alert/notification.

In a closed-loop system, the alarm system can take into account the basal rate adjustments and correction boluses that will be performed based on the generated glucose prediction and adjust the future glucose predictions accordingly. For example, if the closed-loop module will take the appropriate delivery actions that will keep the user in the therapy target range, then the system does not need to alarm the user, and an alarm or notification may not be generated.

In addition, the system does not alarm the user of issues that the system can resolve on its own, such as when the user's glucose is increasing, but predictions show that the closed-loop algorithm will be able to handle and address the rise. The system will also give the user an early warning of issues it predicts will appear in the future, such as when a user's glucose is high, but the user has little insulin remaining in his/her reservoir. Thus, embodiments of the inventions herein provide a significant advantage over existing systems and methodologies by providing a reduction in the number of non-actionable alarms.

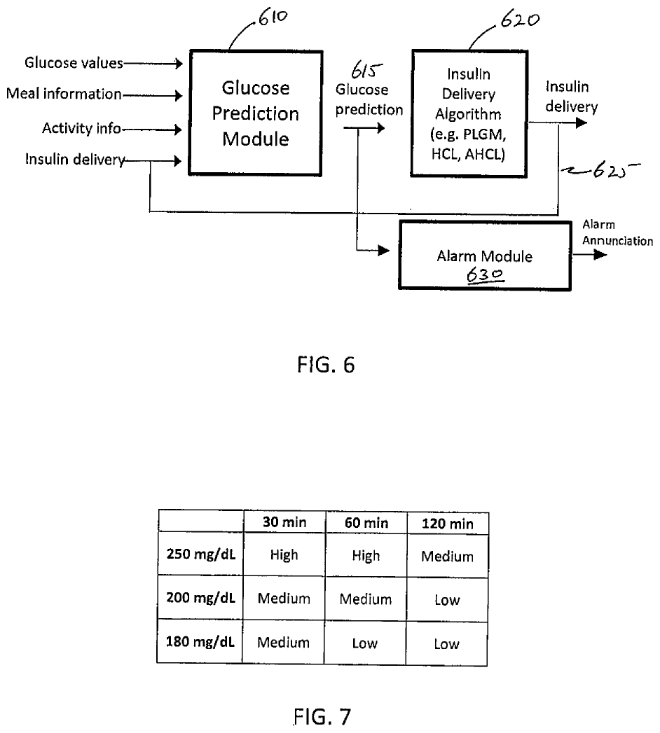

In embodiments of the inventions herein, the predictions can be computed in predetermined intervals. Thus, in one embodiment, the predictions may be computed in 5-minute intervals for a fixed time horizon, such as, e.g., 2 hours. However, the time horizon can be longer if a more reliable prediction method is used, or vary depending on conditions. In addition, in embodiments of the inventions herein, the predicted glucose values are evaluated in relation to pre-defined high and low glucose thresholds, which can be fixed or user-settable. The system determines whether the user's glucose is predicted to reach one of these thresholds, and if it is, how soon. Once this information is obtained, the system proceeds to determine the priority of an alarm based on this information. For example, in one embodiment, the system may consider a mapping of alarm priority values based on how severe the user state is, and how much time the user has spent in that state. In this regard, the table in FIG. 7 provides a matrix that demonstrates one such mapping for an example high-glucose alarm.

As noted hereinabove, in embodiments of the invention, the pre-defined high and low glucose thresholds may be fixed, or may be user-settable. In addition, the time periods can be user-settable to vary the behavior of the notification system. This can be done either by allowing the user to directly set each level (a type of an “advanced mode”), or by providing several pre-defined combinations to allow the user to switch between different overall modes (e.g., “aggressive” mode, “quiet” mode, “meeting” mode, etc.). The alarm system is a dynamic system, in which the alerts can be auto resolved and change category automatically based on the current predicted state.

Thus, as described, embodiments of the inventions herein enable generation of real-time simulations of the user's glucose trend not just based on the state of the user, but also based on how the system will react to this state. Moreover, these simulations are computed using a comprehensive list of factors affecting the user's glucose levels. In this way, the inventive intelligent alarm system drastically improves user experience of insulin pump systems by giving the user notifications which are much more actionable and context-aware than existing methodologies that use simple thresholds and glucose data in isolation. In addition, by simulating insulin delivery along with glucose dynamics, the inventive systems and methodologies can predict the behavior of the system and warn the user of potential issues earlier. This, in turn, allows the user to address these issues in early stages, before they become severe.

By providing the user with notifications that are much more actionable and context-aware, the inventive intelligent alarm systems and methods are able to drastically improve user experience of insulin pump systems. In addition, by simulating insulin delivery along with glucose dynamics, the systems and methodologies herein can predict the behavior of the system and warn the user of potential issues earlier which, in turn, provides the user with an opportunity to address these issues in early stages, before they become severe. Moreover, displaying to the user the glucose predictions in a such a way that the user can not only see what his/her glucose value will be in the future, but also how the system will react to it, the user interface is improved. As noted, when the user enters a new input to the system—such as, e.g., a meal entry—the predictions will be updated (and displayed to the user) in real time. In addition, the system can dynamically auto resolve alerts, and/or change category automatically, based on the current predicted state.

While the description above refers to particular embodiments of the present inventions, it will be understood that many modifications may be made without departing from the spirit thereof. Additional steps and changes to the order of the algorithms can be made while still performing the key teachings of the present inventions. Thus, the accompanying claims are intended to cover such modifications as would fall within the true scope and spirit of the present inventions. The presently disclosed embodiments are, therefore, to be considered in all respects as illustrative and not restrictive, the scope of the inventions being indicated by the appended claims rather than the foregoing description. All changes that come within the meaning of, and range of, equivalency of the claims are intended to be embraced therein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, or may be added or merged. In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

1-21. (canceled)

22. A method of providing an actionable notification to a user of a glucose management system including an infusion device and a sensor device, the method comprising:
measuring, by the sensor device, a glucose level of the user of the glucose management system;
generating, by the infusion device, a predicted glucose profile for the user based on the glucose level and based on meal information, activity information, and insulin information;
determining, by the infusion device, whether the predicted glucose profile is expected to reach an acceptable level within an expected time window, wherein the predicted glucose profile is expected to reach an acceptable level when the user's glucose level is expected to reach a therapy target range within the expected time window;
making a determination, by the infusion device, as to whether a notification should be generated to alert the user when the predicted glucose profile is not expected to reach an acceptable level within the expected time window; and
providing, by the infusion device, the notification to the user when the notification is generated.

23. The method of claim 22, further comprising storing glucose levels over time, and wherein generating the predicted glucose profile for the user includes generating the predicted glucose profile based on both the glucose level and one or more glucose levels stored over time.

24. The method of claim 22, wherein the meal information includes carbohydrate amount information for one or more of a previously consumed meal, a planned meal, and a meal currently being consumed by the user.

25. The method of claim 22, wherein the meal information includes one or more of a time at which a meal was consumed by the user and a time at which a planned meal is expected to be consumed by the user.

26. The method of claim 22, wherein the activity information includes one or more of a type and duration of exercise previously engaged in by the user, expected to be engaged in by the user, and currently being engaged in by the user.

27. The method of claim 22, wherein the insulin information includes an amount of insulin that was previously delivered to the user, an amount of insulin currently being delivered to the user, or both.

28. The method of claim 22, wherein the insulin information includes an insulin delivery pattern.

29. The method of claim 22, wherein the glucose management system further includes a display.

30. The method of claim 29, further comprising displaying the predicted glucose profile to the user on the display.

31. The method of claim 29, further comprising displaying, in real time on the display, trending information for the predicted glucose profile.

32. The method of claim 29, wherein providing the notification to the user includes displaying the notification to the user on the display, sounding an alarm, or both.

33. The method of claim 22, wherein generating the predicted glucose profile for the user includes generating the predicted glucose profile based on the current state of the glucose management system.

34. The method of claim 33, wherein the current state of the glucose management system includes one or more of the sensor device's calibration status, the sensor device's connection status, and a battery level of the glucose management system.

35. The method of claim 33, wherein the current state of the glucose management system includes an amount of insulin remaining in the infusion device.

36. The method of claim 33, wherein the current state of the glucose management system includes sensor reliability metrics.

37. The method of claim 22, wherein the infusion device is an insulin pump, and the glucose management system is a closed-loop system.

38. The method of claim 22, further comprising generating a plurality of predicted glucose profiles, wherein each predicted glucose profile of the plurality of predicted glucose profiles corresponds to a respective predetermined time interval within the expected time window.

39. The method of claim 22, further comprising calculating an expected amount of insulin to be delivered to the user based on the predicted glucose profile, and wherein the determination as to whether the notification should be generated is additionally made based on the expected amount of insulin to be delivered to the user.

40. The method of claim 22, further comprising assigning a priority value to the notification based on the predicted glucose profile.

41. The method of claim 40, herein the priority value is periodically reevaluated.

42. A glucose management system including an infusion device and a sensor device, the glucose management system configured to perform the method of claim 22.
